(19) United States
(12) Patent Application Publication
(10) Pub. No.: US 2026/0364310 A1
(43) Pub. Date: Nov. 19, 2026

(54) TENSILE STRENGTH DEVICE

(71) Applicant: MicroVention, Inc., Aliso Viejo, CA (US)

(72) Inventors: Jeffrey Inlow, San Clemente, CA (US); Veronica Romo, Gilroy, CA (US); Jennifer Reyes, Irvine, CA (US)

(73) Assignee: MicroVention, Inc., Aliso Viejo, CA (US)

(21) Appl. No.: 19/488,378

(22) PCT Filed: May 31, 2024
(86) PCT No.: PCT/US2024/032044

§ 371 Date: Nov. 26, 2025

Related U.S. Application Data
(60) Provisional application No. 63/505,386, filed on May 31, 2023.

(51) Int. Cl. A61M 25/01; A61M 25/00

(52) U.S. Cl. CPC A61M 25/0138; A61M 25/001; A61M 25/0108; A61M 2025/015; A61M 2205/0266

(57) ABSTRACT

A tensile strength device for increasing tensile strength of a distal tip of a medical device while maintaining sufficient flexibility for the medical device to traverse through tortuous anatomies. The tensile strength device may include a base component and a wire wound around the base component to form a coil. A distal end of the tensile strength device may include an adhesive which both blunts the end of the tensile strength device to prevent puncture and secures the distal end of the coil to the base component. The base component may be largely omitted, with a distal length of the wire forming the coil turning back to extend through an inner diameter of the coil to function as the base component. A stopper may be positioned at a proximal end of the tensile strength device to form an interference lock with the medical device.

### RELATED APPLICATIONS

This application claims benefit of and priority to U.S. Patent Application Ser. No. 63/505,386 filed May 31, 2023 and entitled Tensile Strength Device, which is hereby incorporated herein by reference in its entirety.

### BACKGROUND OF THE INVENTION

Various types of medical devices benefit flexibility at or near their distal tips. Such flexibility may be useful for traversing tortuous anatomies, such as the aortic arch or other tortuous vessels. However, such flexibility may also come at the cost of tensile strength, which can affect the overall structural integrity of such medical devices.

It would be desirable to increase tensile strength in the distal tip of a medical device while maintaining sufficient flexibility of the medical device to traverse tortuous anatomies.

### SUMMARY OF THE INVENTION

In some aspects, a medical device includes a tensile strength device at or near its distal end for increasing tensile strength in the distal tip of the medical device while maintaining sufficient flexibility of the medical device to traverse tortuous anatomies.

In some aspects, the tensile strength device may comprise a base component and at least one wire wound around the base to form a coil.

In some aspects, the base component of the tensile strength device may be composed of a different material than the coil.

In some aspects, the base component of the tensile strength device may be composed of a shape-memory-material.

In some aspects, the base component of the tensile strength device may be composed of a nickel-titanium alloy such as Nitinol.

In some aspects, the coil may be composed of a radiopaque material.

In some aspects, the coil may be composed of tantalum.

In some aspects, the coil may be composed of tungsten.

In some aspects, the coil may be composed of gold.

In some aspects, the coil may be composed of platinum.

In some aspects, the coil may be composed of palladium.

In some aspects, the coil may be composed of a metallic alloy.

In some aspects, a ball may be formed at a distal end of the tensile strength device.

In some aspects, the ball may be composed of an adhesive material.

In some aspects, a proximal end of the tensile strength device may be comprised of a stopper. The stopper may be comprised of a ball or sphere. The outer diameter of the ball or sphere may be greater than an internal diameter of the medical device.

In some aspects, the tensile strength device may be integral with the medical device.

In some aspects, both the medical device and the tensile strength device may be integrally formed from a unitary body.

In some aspects, both the medical device and the tensile strength device may be laser cut together.

In some aspects, the tensile strength device may be attached to the medical device, either removably or fixedly.

In some aspects, the tensile strength device may comprise a radiopaque marker.

In some aspects, the radiopaque marker may comprise a flange composed of a radiopaque material.

In some aspects, the radiopaque marker may comprise a band such as a circular ring.

In some aspects, the tensile strength device may be comprised of a single wire which is wound to form a coil.

In some aspects, a portion of the single wire may extend linearly through the coil from the distal end to the proximal end of the tensile strength component.

In some aspects, a portion of the single wire may extend through an entire length of an interior of the coil.

In some aspects, a portion of the single wire may extend through only a portion of the entire length of the interior of the coil.

In some aspects, the techniques described herein may relate to a tensile strength device, including: a base component including a proximal end and a distal end; and a wire wound around the base component to form a coil, the coil being positioned around at least a length of the base component between the proximal end and the distal end of the base component.

In some aspects, the techniques described herein relate to a tensile strength device, wherein the base component is composed of Nitinol.

In some aspects, the techniques described herein relate to a tensile strength device, wherein the coil is composed of a radiopaque material.

In some aspects, the techniques described herein relate to a tensile strength device, wherein the coil is composed of platinum, tungsten, palladium, gold, or alloys thereof.

In some aspects, the techniques described herein relate to a tensile strength device, further including a radiopaque marker positioned at or near a proximal end of the coil.

In some aspects, the techniques described herein relate to a tensile strength device, wherein the radiopaque marker includes a flange extending at least partially around the base component.

In some aspects, the techniques described herein relate to a tensile strength device, wherein the radiopaque marker includes a band.

In some aspects, the techniques described herein relate to a tensile strength device, wherein the distal end of the base component includes an opening.

In some aspects, the techniques described herein relate to a tensile strength device, wherein a distal end of the coil is secured within the opening.

In some aspects, the techniques described herein relate to a tensile strength device, wherein a distal end of the coil is attached to the base component by an adhesive.

In some aspects, the techniques described herein relate to a tensile strength device, wherein the adhesive is composed of a UV curable glue.

In some aspects, the techniques described herein relate to a tensile strength device, wherein the adhesive is formed into a ball.

In some aspects, the techniques described herein relate to a tensile strength device, further including a stopper attached to or positioned at the proximal end of the base component.

In some aspects, the techniques described herein relate to a tensile strength device, wherein the stopper includes a ball.

In some aspects, the techniques described herein relate to a tensile strength device, wherein the distal end of the base component includes a projection.

In some aspects, the techniques described herein relate to a tensile strength device, wherein the projection extends downwardly from the distal end of the base component.

In some aspects, the techniques described herein relate to a tensile strength device, wherein the projection is rounded or curved.

In some aspects, the techniques described herein relate to a tensile strength device, wherein the projection includes an opening.

In some aspects, the techniques described herein relate to a tensile strength device, further including an adhesive at least partially encapsulating the projection.

In some aspects, the techniques described herein relate to a tensile strength device, including: a wire wound to form a coil, wherein a distal length of the wire is turned so as to extend linearly through an inner diameter of the coil; a radiopaque marker attached to a proximal end of the coil; and a stopper attached to the radiopaque marker.

In some aspects, the techniques described herein relate to a medical device, including: a tubular member including a distal end; and a tensile strength device attached to the distal end of the tubular member, the tensile strength device being included of: a base component including a proximal end and a distal end; and a wire wound around the base component to form a coil, the coil being positioned around at least a length of the base component between the proximal end and the distal end of the base component.

In some aspects, the techniques described herein relate to a method of increasing tensile strength of a medical device, including: attaching a tensile strength device to a distal end of the medical device, the tensile strength device being included of: a base component including a proximal end and a distal end; and a wire wound around the base component to form a coil, the coil being positioned around at least a length of the base component between the proximal end and the distal end of the base component.

### BRIEF DESCRIPTION OF THE DRAWINGS

The following figures are included to illustrate certain example aspects of the present disclosure and should not be viewed as exclusive or limiting. The subject matter disclosed is capable of considerable modifications, alterations, combinations, and equivalents in form and function, as will occur to one having ordinary skill in the art and having the benefit of this disclosure. The present disclosure references the drawings as follows:

FIG. 1A illustrates a side view of a tensile strength device according to one example.

FIG. 1B illustrates a side view of a tensile strength device according to one example.

FIG. 2 illustrates a side view of a base component of a tensile strength device according to one example.

FIG. 3 illustrates a side view of a medical device including a tensile strength device according to one example.

FIG. 4A illustrates a side view of a tensile strength device connected to a medical device according to one example.

FIG. 4B illustrates a side view of a tensile strength device connected to a medical device according to one example.

FIG. 5 illustrates a side view of a tensile strength device according to one example.

### DETAILED DESCRIPTION

It will be appreciated by persons skilled in the art that the present disclosure is not limited to what has been particularly shown and described herein. A variety of modifications and variations are possible in view of the teachings herein without departing their scope, spirit, or intent.

While different examples may be described in this specification, it is specifically contemplated that any of the features from the different examples can be used and brought together in any combination. In other words, the features of different examples can be mixed and matched with each other. Hence, while every permutation of features from different examples may not be explicitly shown or described, it is the intention of this disclosure to cover any such combinations, especially as may be appreciated by one of skill in the art.

Specific aspects of the invention will now be described with reference to the accompanying drawings. This invention may, however, be embodied in many different forms and should not be construed as limited to the examples set forth herein; rather, these examples are provided so that this disclosure will be thorough and complete, and will fully convey the scope of the invention to those skilled in the art. The terminology used in the detailed description of the examples illustrated in the accompanying drawings is not intended to be limiting of the invention. In the drawings, like numbers refer to like elements.

The terminology used in this disclosure should be interpreted in a permissive manner and is not intended to be limiting. In the drawings, like numbers refer to like elements. Unless otherwise noted, all of the accompanying drawings are not to scale. Unless otherwise noted, the term “about” is defined to mean plus-or-minus 10% of a stated value, inclusive.

The terms distal or distally generally refer to a direction or area towards an end of a device within a patient (e.g., away from a physician/clinician), while the terms proximal or proximally refer to a direction or area toward an end of a device that remains outside of a patient (e.g., toward or closer to a physician/clinician or handle/hub of a device).

Disclosed herein are various examples of a tensile strength device which may be integrated with or attached to various medical devices so as to improve tensile strength at the distal tip of such medical devices while maintaining sufficient flexibility of the overall medical devices to traverse tortuous anatomies. It should be appreciated that the methods and devices shown and/or described herein may be utilized with a wide range of medical devices.

By way of example, any medical device which may traverse through a tortuous anatomy may benefit from the methods and devices shown and/or described herein. As a more specific but non-limiting example, such medical devices may include various thrombectomy devices, occlusion devices, tubular devices, clot retrieval devices, embolic protection devices, and the like. The medical device may comprise a hypotube, with the tensile strength device being attached to a distal end of the hypotube.

The medical device may be adjustable between a collapsed configuration (e.g., for delivery within a delivery device) and an expanded configuration (e.g., for use). When in the expanded configuration, the medical device may comprise, for example, a plurality of enlarged bulbs, with the tensile strength device 100 being attached to or extending from the distal-most of such enlarged bulbs.

Further, the methods and devices shown and/or described herein may be utilized with laser cut medical devices, braided medical devices, woven medical devices, and/or any other configuration of medical device which may benefit from increased tensile strength at its distal tip while maintaining overall flexibility (e.g., without a significant impact on the overall flexibility of the medical device).

Additionally, the use of a tensile strength device as disclosed herein may improve visualization of the distal end of the medical device while reducing the risk of injury, such as due to puncturing of an anatomy.

The tensile strength device may comprise a base component having a proximal end and a distal end. The base component may comprise a substantially elongated configuration such as, for example, an elongated member. The base component may be substantially flat. The cross-section of the base component may vary. For example, the cross-section of the base component may be circular, oval-shaped, triangular, rectangular, square-shaped, and the like. A substantial length of the tensile strength device may be linear.

The distal end or portion of the base component of the tensile strength device may comprise a downward projection. The downward projection may comprise a substantially rounded shape. The downward projection may include an opening formed therein. The shape, size, orientation, and positioning of the opening may vary. A coil, such as its distal end or distal portion, may be secured or looped within the opening as discussed in more detail below.

The base component of the tensile strength device may be comprised of various shapes, sizes, and materials. The base component of the tensile strength device may be thin enough for required flexibility, but thick enough to impart a desired tensile strength. The base component of the tensile strength component may be the same material as the overall medical device from which it extends or may be a different material than the overall medical device from which it extends.

The base component of the tensile strength device may be composed of a shape-memory-material. The base component of the tensile strength device may be composed of Nitinol.

The distal end of the tensile strength device may be blunt or rounded so as to prevent or reduce the likelihood of a puncture or other undesirable situations during delivery, deployment, implantation, or retrieval of the medical device.

The blunt distal end of the tensile strength device may be formed in various manners, such as by forming a ball or dome at the distal end of the base component of the tensile strength device. The ball may be integral with the tensile strength device or attached thereto, such as by an adhesive. In some examples, the adhesive may be omitted entirely.

By way of example, the ball may be formed from a cured or dried adhesive positioned at least partially around a distal tip or distal portion of the base component. More specifically, the ball may be formed from various UV light curable adhesives, such as UV glue. In examples in which the blunted end of the tensile strength device is formed by an adhesive, the adhesive may also serve the function of securing a coil over the base component. However, in some examples, the adhesive may be utilized only to provide a blunted (as opposed to sharp) tip. In such examples, the coil may be secured to the base component by other methods.

The proximal end of the tensile strength device may include a stopper which may function to secure the tensile strength device to a distal portion or distal end of the medical device, or within a distal portion or distal end of the medical device. The stopper may serve the function of preventing the tensile strength device from becoming dislodged, separated, or other disconnected from the medical device from which it extends. The stopper may comprise a ball or other spherical member having an outer diameter which is larger than the inner diameter of the medical device from which the tensile strength device extends, thereby forming an interference lock.

The manner by which the base component of the tensile strength device may be formed or fabricated may vary. By way of example, the base component of the tensile strength device may be cut from a sheet of a material, such as a superelastic Nitinol sheet. All edges of the tensile strength device may be deburred.

As previously mentioned, the tensile strength device may be attached, such as by direct or indirect connection, to a distal end of a medical device. As one example, the tensile strength device's proximal end may be partially inserted within a channel or cavity at a distal end of the medical device, with the stopper preventing the tensile strength device from becoming prematurely or inadvertently dislodged.

In some examples, the tensile strength device may be integral with the medical device such that the tensile strength device extends from a distal end of the medical device. In such examples, the tensile strength device may be laser cut along with the remainder of the medical device. In such examples, the material of the tensile strength device may be the same as the material of the overall medical device from which it extends.

The tensile strength device may include a radiopaque marker. The positioning of the radiopaque marker may vary in different examples. In some examples, multiple radiopaque markers may be utilized to, e.g., identify the respective ends of the tensile strength device. In an example, the radiopaque marker may be located at or near a distal portion or a distal end of the tensile strength device.

In examples in which a coil is wound around the base component, the radiopaque marker may be positioned at or near the proximal end of such a coil. The proximal end of the coil may be welded or otherwise attached to the base component.

The radiopaque marker may be composed of various radiopaque materials, such as but not limited to gold, platinum, tungsten, palladium, tantalum, alloys of the foregoing, and the like. The radiopaque marker may comprise a band such as an annular or semi-annular ring positioned at least partially around the base component at or near its proximal end.

The tensile strength device may include a wire which may be wound around at least a portion of the length of the base component to form a coil. The coil may be composed of various radiopaque materials, including the aforementioned example radiopaque materials. The coil may be tightly or loosely wound. A distal portion or distal end of the coil may be attached to a distal portion or distal end of the tensile strength device, such as within the opening. The manner by which the coil may be attached to the tensile strength device may vary in different examples. In one example, the coil may be attached to the tensile strength device by an adhesive.

The base component may not be elongated in some examples, but instead may form a base flange from which a coiled wire extends. In such examples, filled wire (e.g., drawn filled tubing) may be used to form the distal coil. In such examples, the coiled wire may be composed of a radiopaque core within an outer jacket. In some examples, the radiopaque core may be composed of any of the aforementioned radiopaque materials, such as but not limited to platinum. The outer jacket may be composed of a Nitinol tube, among other materials. Such a configuration may provide radiopacity from the platinum core and allow welding of the outer Nitinol tube to a medical device which is also composed of Nitinol. The Nitinol-to-Nitinol weld may provide more tensile strength than other welds, such as a tantalum-to-Nitinol weld.

Further, at the distal end of the coil, instead of the filar terminating at the end of the coil, it may be formed to wrap back inside the inner diameter of the coil, either fully along the length of the coil through to the proximal end of the coil or only partially along the length of the coil. Such a filar could be used as a strength-resistant member and/or to provide additional tensile strength if formed into a ball so as to form an interference lock with the inner diameter of the base component as previously discussed.

Specific examples are described further below. However, it should be understood that any of the features from any of the examples can be mixed and matched with each other in any combination. Hence, the present disclosure should not be restricted to only these examples, but any broader combination thereof.

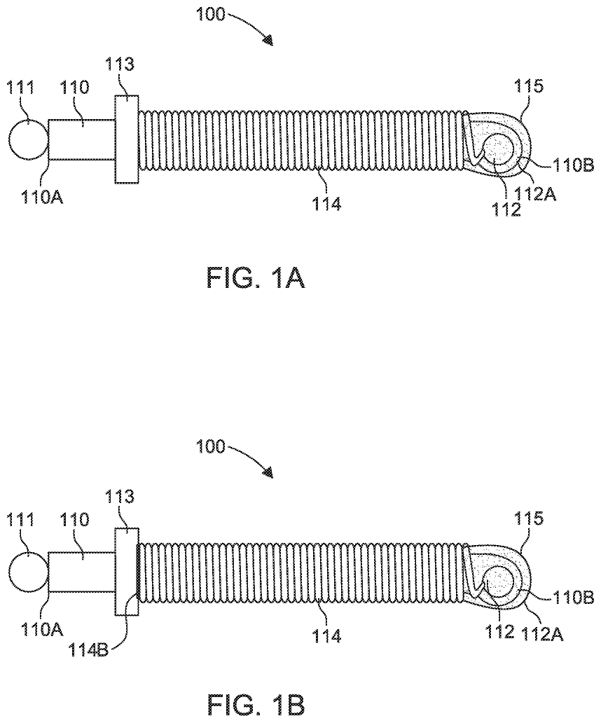

FIG. 1A illustrates a side view of a tensile strength device 100 according to one example. As shown in FIG. 1A, the tensile strength device 100 may comprise a base component 110 having a proximal end 110A and a distal end 110B. The proximal end 110A may be attached to or integrally extend from a distal portion or distal end of a medical device 120.

The proximal end 110A may include a stopper 111 which is integrally formed with or attached to the proximal end 110A of the base component 110. The stopper 111 may comprise a spherical shape as shown or may comprise various other shapes, including rectangular or the like.

The outer diameter or width of the stopper 111 may be greater than an inner diameter or width of the medical device 120 so as to form an interference lock and thereby prevent the tensile strength device 100 becoming detached from the medical device 120. Although the stopper 111 is illustrated as comprising a ball-shaped configuration, various other shapes may be utilized. The stopper 111 may comprise any type of enlargement which would serve the purpose of an interference lock and thus should not be construed as limited by the examples shown in the figures.

Continuing to reference FIG. 1A, it can be seen that a radiopaque marker 113 may be positioned at or near the proximal end 110A of the base component 110. The radiopaque marker 113 may be composed of various radiopaque materials as previously discussed. The radiopaque marker 113 may be comprised of a flange or band which at least partially surrounds the base component 110. The radiopaque marker 113 may be integrally formed with, or attached to, the base component 110.

FIG. 1A also illustrates a coil 114 which may be wound around at least a portion of the length of the base component 110. The coil 114 may be formed from a wire 114A which may be wound around the base component 110 as shown. The wire 114A forming the coil 114 may be comprised of a radiopaque material. In some examples, the coil 114 may be formed from multiple wires 114A.

Although FIG. 1A illustrates that the coil 114 may be tightly wound, it should be appreciated that the coil 114 may be more loosely wound than shown in the illustrated example. The proximal end of the coil 114 may terminate at the radiopaque marker 113 as shown. The manner by which the coil 114 is wound, including the pitch of the coil 114 and the presence and/or size of any gaps between windings of the coil 114, may impact stiffness of the coil 114. Variations of these attributes may thus be utilized to fine-tune the specifications of the tensile strength device 100 to tailor the overall stiffness of the tensile strength device 100 to particular preferences of users, medical procedures, and/or medical devices 120. In some examples, the coil 114 may be tightly wound so as to minimize or eliminate gaps between windings of the coil 114.

A distal end of the coil 114 may be attached or otherwise secured to the distal end 110B of the base component 110. In some examples, the distal end of the coil 114 may simply be tied to or looped around the distal end 110B of the base component 110. In other example examples, such as shown in FIG. 1A, the distal end of the coil 114 may be secured to the distal end 110B of the base component 110 by an adhesive 115. The adhesive 115 may be formed into an enlargement, such as substantially a ball shape, which may serve the function of blunting the end of the base component 110 and thereby preventing puncture of a vessel wall.

FIG. 1B illustrates a side view of a tensile strength device 100 according to one example. It should be appreciated that the manner by which a proximal end or proximal portion of the coil 114 may be attached to the base component 110 or radiopaque marker 113 may vary. In the example shown in FIG. 1B, it can be seen that the proximal end or proximal portion of the coil 114 may be attached to the base component 110 or radiopaque marker 113 by a weld 114B. The weld 114B may function to prevent movement and provide additional security, rigidity, and structural integrity of the tensile strength device 100. Various types of welds 114B may be utilized, including but not limited to laser welds.

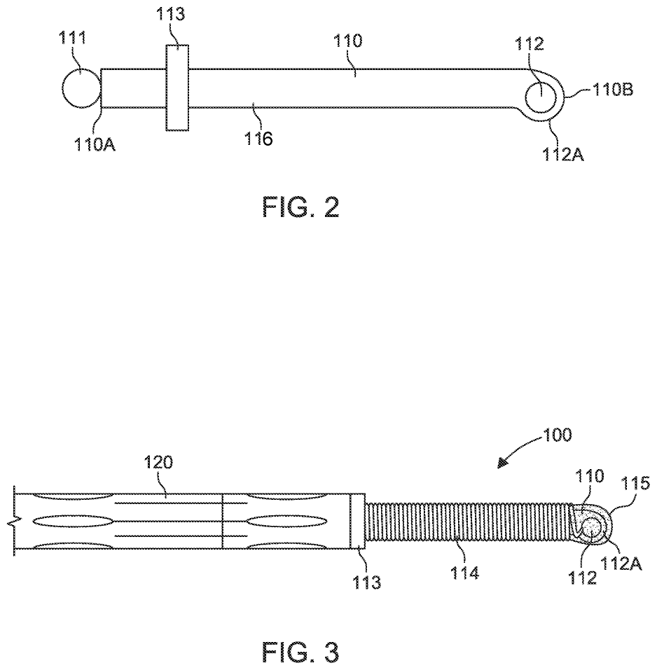

FIG. 2 illustrates a side view of a base component 110 of a tensile strength device 100 according to one example. In the example shown in FIG. 2, the coil 114 has been omitted to improve visualization of the base component 110. As shown, the distal end 110B of the base component 110 may include an opening 112 in which the distal end of the coil 114 may be secured or attached.

The base component 110 may comprise an elongated portion 116 and a downward projection 112A extending from a distal end of the elongated portion 116 as shown in FIG. 2. The elongated portion 116 and downward projection 112A may be integrally formed from a unitary body or may comprise different components that are interconnected, such as by welding, fasteners, or the like.

The distal end 110B of the base component 110 may comprise a downward projection 112A as shown in FIGS. 1A-1B, with the downward projection 112A having a substantially rounded or curved configuration. The size of the opening 112 may vary in different examples and thus should not be construed as limited in scope by the example shown in FIG. 2 or any other figure.

The overall dimensions of the tensile strength device 100, including respective dimensions of the base component 110, elongated portion 116, downward projection 112A, and opening 112, may vary in different examples to suit different medical devices 120. In an example, a length of the base component 110 from its proximal end 110A to the center of the opening 112 may comprise between about 0.15 inch and 0.35 inch. In an example, the elongated portion 116 of the base component 110 may comprise a tubular body having an inner lumen diameter of between about 0.005 inch and 0.007 inch and an overall outer diameter of between about 0.008 inch and 0.012 inch.

In an example, the diameter of the downward projection 112A may comprise between about 0.007 inch and about 0.009 inch. In an example, diameter of the opening 112 may comprise between about 0.0035 inch and 0.0045 inch. Thus, the opening 112 may have a diameter that is about half of the overall diameter of the downward projection 112A.

FIG. 3 illustrates a side view of a medical device 120 including a tensile strength device 100 according to one example. As shown, the tensile strength device 100 may be attached to or may extend from the distal end of the medical device 120.

FIG. 3 illustrates the medical device 120 in a substantially collapsed position. In some examples in which the medical device 120 may comprise a tubular member, the proximal portion or proximal end 110A of the base component 110 may be partially inserted within a channel or cavity at the distal end or distal portion of the medical device 120. The stopper 111 may aid in securing the base component 110 in such a manner and preventing the tensile strength device 100 from dislodging or disengaging from the medical device 120.

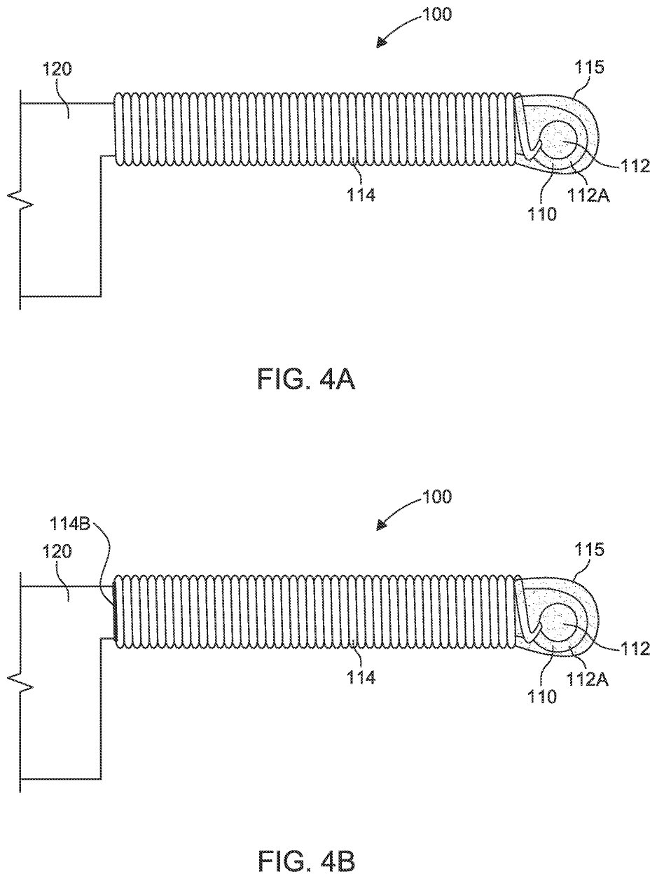

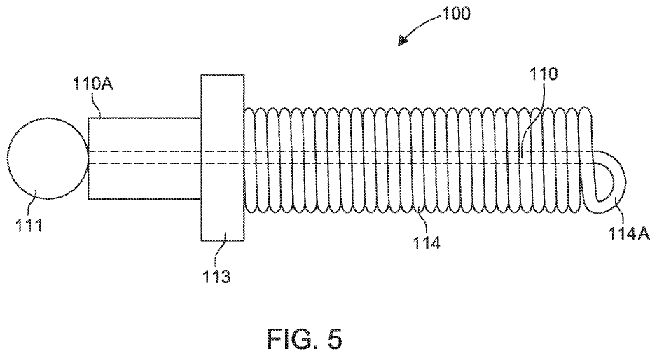

FIG. 4A illustrates a side view of a tensile strength device 100 connected to a medical device 120 according to one example. As previously discussed, the tensile strength device 100 may be integrally formed with the medical device 120 such that the tensile strength device 100 may extend distally from a distal end of the medical device 120, such as shown in FIG. 4A. By way of example, the tensile strength device 100 may be laser cut integrally with the medical device 120.

FIG. 4B illustrates a side view of a tensile strength device 100 connected to a medical device 120 according to one example. It should be appreciated that the manner by which a proximal end or proximal portion of the coil 114 may be attached to the tensile strength device 100, which in this example extends integrally from the medical device 100, may vary. In the example shown in FIG. 4B, it can be seen that the proximal end or proximal portion of the coil 114 may be attached to the tensile strength device 100 by a weld 114B. The weld 114B may function to prevent movement and provide additional security, rigidity, and structural integrity of the tensile strength device 100.

FIG. 5 illustrates a side view of a tensile strength device 100 according to one example. In the example of FIG. 5, it can be seen that the base component 110 may be largely omitted, with a length of the wire 114A forming the coil 114 being turned back through the inner diameter of the coil 114 to function as the base component 110. In this manner, the tensile strength device 100 may be substantially formed from the wire 114A.

The tensile strength device 100 may nonetheless include a radiopaque marker 113 positioned at or near a proximal end of the coil 114. The tensile strength device 100 may also include a stopper 111 for forming an interference lock with the medical device 120, though the stopper 111 may be omitted in certain examples, such as examples in which the tensile strength device 100 may be integrally formed with the medical device 120.

### Clauses

Clause 1. A tensile strength device, comprising: a base component comprising a proximal end and a distal end; and a wire wound around the base component to form a coil, the coil being positioned around at least a length of the base component between the proximal end and the distal end of the base component.
Clause 2. The tensile strength device of clause 1, wherein the base component is composed of Nitinol.
Clause 3. The tensile strength device of clause 2, wherein the coil is composed of a radiopaque material.
Clause 4. The tensile strength device of clause 1, wherein the coil is composed of platinum, tungsten, palladium, gold, or alloys thereof.
Clause 5. The tensile strength device of clause 1, further comprising a radiopaque marker positioned at or near a proximal end of the coil.
Clause 6. The tensile strength device of clause 5, wherein the radiopaque marker is comprised of a flange extending at least partially around the base component.
Clause 7. The tensile strength device of clause 5, wherein the radiopaque marker is comprised of a band.
Clause 8. The tensile strength device of clause 1, wherein the distal end of the base component comprises an opening.
Clause 9. The tensile strength device of clause 8, wherein a distal end of the coil is secured within the opening.
Clause 10. The tensile strength device of clause 1, wherein a distal end of the coil is attached to the base component by an adhesive.
Clause 11. The tensile strength device of clause 10, wherein the adhesive is composed of a UV curable glue.
Clause 12. The tensile strength device of clause 10, wherein the adhesive is formed into a ball.
Clause 13. The tensile strength device of clause 1, further comprising a stopper attached to or positioned at the proximal end of the base component.
Clause 14. The tensile strength device of clause 13, wherein the stopper is comprised of a ball.
Clause 15. The tensile strength device of clause 1, wherein the distal end of the base component is comprised of a projection.
Clause 16. The tensile strength device of clause 15, wherein the projection extends downwardly from the distal end of the base component.
Clause 17. The tensile strength device of clause 15, wherein the projection is rounded or curved.
Clause 18. The tensile strength device of clause 15, wherein the projection includes an opening.
Clause 19. The tensile strength device of clause 15, further comprising an adhesive at least partially encapsulating the projection.
Clause 20. A tensile strength device, comprising: a wire wound to form a coil, wherein a distal length of the wire is turned so as to extend linearly through an inner diameter of the coil; a radiopaque marker attached to a proximal end of the coil; and a stopper attached to the radiopaque marker.
Clause 21. A medical device, comprising: a tubular member including a distal end; and a tensile strength device attached to the distal end of the tubular member, the tensile strength device being comprised of: a base component comprising a proximal end and a distal end; and a wire wound around the base component to form a coil, the coil being positioned around at least a length of the base component between the proximal end and the distal end of the base component.
Clause 22. A method of increasing tensile strength of a medical device, comprising: attaching a tensile strength device to a distal end of the medical device, the tensile strength device being comprised of: a base component comprising a proximal end and a distal end; and a wire wound around the base component to form a coil, the coil being positioned around at least a length of the base component between the proximal end and the distal end of the base component.

Although the invention has been described in terms of particular examples and applications, one of ordinary skill in the art, in light of this teaching, can generate additional examples and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1-22. (canceled)

23. A tensile strength device, comprising:
a base component comprising a proximal end and a distal end; and
a wire wound around the base component to form a coil, the coil being positioned around at least a length of the base component between the proximal end and the distal end of the base component.

24. The tensile strength device of claim 23, wherein the base component is composed of Nitinol.

25. The tensile strength device of claim 24, wherein the coil is composed of a radiopaque material.

26. The tensile strength device of claim 23, further comprising a radiopaque marker positioned at or near a proximal end of the coil.

27. The tensile strength device of claim 26, wherein the radiopaque marker is comprised of a flange extending at least partially around the base component.

28. The tensile strength device of claim 26, wherein the radiopaque marker is comprised of a band.

29. The tensile strength device of claim 23, wherein the distal end of the base component comprises an opening.

30. The tensile strength device of claim 29, wherein a distal end of the coil is secured within the opening.

31. The tensile strength device of claim 23, wherein a distal end of the coil is attached to the base component by an adhesive.

32. The tensile strength device of claim 31, wherein the adhesive is composed of a UV curable glue.

33. The tensile strength device of claim 31, wherein the adhesive is formed into a ball.

34. The tensile strength device of claim 23, further comprising a stopper attached to or positioned at the proximal end of the base component.

35. The tensile strength device of claim 34, wherein the stopper is comprised of a ball.

36. The tensile strength device of claim 23, wherein the distal end of the base component is comprised of a projection.

37. The tensile strength device of claim 36, wherein the projection extends downwardly from the distal end of the base component.

38. The tensile strength device of claim 36, wherein the projection is rounded or curved.

39. The tensile strength device of claim 36, wherein the projection includes an opening.

40. The tensile strength device of claim 36, further comprising an adhesive at least partially encapsulating the projection.

41. A tensile strength device, comprising:
a wire wound to form a coil, wherein a distal length of the wire is turned so as to extend linearly through an inner diameter of the coil;
a radiopaque marker attached to a proximal end of the coil; and
a stopper attached to the radiopaque marker.

42. A medical device, comprising:
a tubular member including a distal end; and
a tensile strength device attached to the distal end of the tubular member, the tensile strength device being comprised of:
a base component comprising a proximal end and a distal end; and
a wire wound around the base component to form a coil, the coil being positioned around at least a length of the base component between the proximal end and the distal end of the base component.
